(19) United States
(12) Patent Application Publication
(10) Pub. No.: US 2024/0016370 A1
(43) Pub. Date: Jan. 18, 2024

(54) ENDODEVICE

(71) Applicant: UNIVERSITÄT BASEL, Basel (CH)

(72) Inventors: Lorin FASEL, Basel (CH); Nicolas GERIG, Basel (CH); Georg RAUTER, Eiken (CH)

(21) Appl. No.: 18/251,450

(22) PCT Filed: Nov. 5, 2021
(86) PCT No.: PCT/EP2021/080722

§ 371 Date: May 2, 2023

(30) Foreign Application Priority Data
Nov. 6, 2020 (CH) 01430/20

(51) Int. Cl. A61B 1/005; A61B 1/00; A61B 1/008; A61B 90/00

(52) U.S. Cl. CPC A61B 1/0052; A61B 1/00006; A61B 1/0057; A61B 1/008; A61B 90/06; A61B 2090/064

(57) ABSTRACT

An endodevice is disclosed with tension control. The endodevice includes a back end unit with an endoscopic unit coupled thereto. The back end unit includes first and second sensor elements, wherein the first sensor element is arranged between a drive member and a first spring element and the second sensor element is arranged between another end of the first spring element and a first tendon coupler. Each of the first and second sensor elements are configured to generate a sensor signal wherein the first sensor element, the second sensor element and the drive member are connected to a control unit. The control unit is configured to obtain sensor signals from the first sensor element and the second sensor element and to compute a deflection of the first spring element on the basis of the sensor signals obtained from the first sensor element and the second sensor element.

### TECHNICAL FIELD

The present invention relates to an endodevice with tension control according to the preamble of independent claim 1.

### BACKGROUND ART

Over the last decades, surgeons have aimed at reducing the size and number of skin incisions required for an intervention. Although these minimally invasive surgeries (MIS) bring many benefits to the patient, it is complicated and often unintuitive for the surgeon to manually manipulate the surgical instruments through a small opening in the skin. Introducing robots to assist with MIS could make instrument manipulation easier, which would allow the surgeon to focus on the actual surgical tasks. One possible setup for robot-assisted surgeries features the robot at the operating table and the surgeon controlling it from a telemanipulation console, therefore eliminating the direct mechanical connection between surgeon and surgical instruments.

In general, the main advantage of telemanipulation over manual MIS is precise motion control of the surgical instruments (e.g., by filtering tremor and motion scaling). However, to steer the surgical instruments, the surgeon currently relies solely on visual feedback but does generally not feel the interaction with the tissue. In comparison to conventional surgeries, this lack of haptic feedback makes it challenging to perform certain surgical tasks (e.g., palpation) despite high performance in device positioning. In other situations, the lack of haptic feedback might even pose a safety threat, e.g., when collisions between the surgical instruments and the surrounding tissue occur outside the visual field of the endoscope camera.

The lack of haptic feedback is thus considered as one of the main drawbacks of current robotic surgical systems. However, different challenges obstruct the implementation of haptic feedback into robotic surgical systems. Haptic perception is not a passive, unidirectional perception like vision or hearing, but it always involves the exchange of mechanical energy in two directions. Because of this bidirectionality, it is difficult to achieve stable control for devices with haptic feedback. Furthermore, it is necessary to measure contact forces between the endoscope and the tissue, but embedding force, torque, or pressure sensors into the endoscope tip has proven to be difficult. Sensors would need to be small, reliable, and able to withstand sterilization procedures.

One approach is to measure the tendon elongation and to use Hooke's Law to estimate the tendon tension. An algorithm was proposed to control the position of the end-effector tip by varying the estimated tendon tension, as described in U.S. Pat. No. 9,101,379 B2.

In US 2012/0123441 A1 for example, control systems and methods for a medical instrument are described which use measurements to determine and control the tensions that actuators apply through instrument transmission systems. The use of tension and feedback allows control of a medical instrument having transmission systems that provide non-negligible compliance between joints and actuators even when the positions of joints cannot be directly related to actuator positions. One embodiment determines joint torques and tensions from differences between desired and measured joint positions. Another embodiment determines joint torques and tensions from differences between desired and measured positions of a tip of the instrument. Determination of tensions from joint torques can be performed using sequential evaluation of joints in an order from a distal end of the instrument toward a proximal end of the instrument.

However, such an approach again creates the need to place a position sensor at the distal end of the endoscope.

It is therefore the object of the present invention to overcome the above-described shortcomings and provide for a structure where all relevant components for determining tension are removed from the instrument portion which is provided for entering the patient's body or any other difficult to access area and by means of which a highly precise shape sensing and thus a highly fine-tuned haptic feedback may be provided at the same time.

### DISCLOSURE OF THE INVENTION

According to the invention these needs are settled by an endodevice as it is defined by the features of independent claim 1. Preferred embodiments are subject of the dependent claims.

In one aspect, the present invention is an endodevice with tension control which comprises a back end unit and an endoscopic unit configured to be coupled to the back end unit. The endoscopic unit includes an endoscopic coupling structure, a bendable rod element and a first tendon and a second tendon arranged to bend a first portion of the bendable rod element and a second portion of the bendable rod element relative to one another. The back end unit includes a back end coupling structure corresponding to the endoscopic coupling structure, a first tendon coupler and a second tendon coupler, a first spring element having a first function end and a second function end, a drive member and a drive or holding member. Thereby, the first spring element is connectable at its first function end to the first tendon via the first tendon coupler and the first spring element is connected to the drive member at its second function end, wherein the drive or holding member is connectable to the second tendon via the second tendon coupler. The back end unit further comprises a first sensor element and a second sensor element, wherein between the drive member and the second function end of the first spring element, the first sensor element is arranged and between the first function end of the first spring element and the first tendon coupler the second sensor element is arranged. Thereby, each of the first sensor element and the second sensor element is configured to generate a sensor signal, wherein the first sensor element, the second sensor element and the drive member are connected to a control unit. The control unit is configured to obtain sensor signals from the first sensor element and the second sensor element and to compute a deflection of the first spring element on the basis of the sensor signals obtained from the first sensor element and the second sensor element.

Primarily, the term “endodevice” in connection with the present invention relates to a medical device which is arranged or embodied to be introduced into a body or body lumen and to be advanced through the body or body lumen to a target location where the intervention is to be executed. Thereby, the term “in the body” or “inside the body” can mean any location in the human or animal body and particularly a quasi embedded location which is not directly accessible from the outside. For example, in the body can mean in between different tissues of the body, such as in between a bone and its surrounding tissue, or inside a body lumen. The term “body lumen” can relate to an inside space of a tubular structure in a human or animal body or to a cavity inside the human or animal body. For example, the body lumen can be a vascular vessel, such as a vein or an artery or a coronary or intracranial vessel or a heart valve, or a tract of a gastrointestinal organ such as stomach or colon, or a region of urinary collecting ducts or of renal tubes, or an interior space of joint, or a mouth or ear, or a combination thereof. The endodevice can be or comprise a rigid or particularly a flexible endoscope, a catheter, a laparoscope, a colonoscope or a similar arrangement.

However, the term “endodevice” in connection with the present invention may also relate to a technical device or technical equipment which is arranged or embodied to carry out inspections and/or maintenance in areas which are likewise difficult to access.

The term “spring element” as used herein may comprise any element that stores and releases energy with small energy loss. The spring element comprises a first and a second “function end”, wherein the function ends represent those portions or sections of the spring element which are operatively connected to those components which cause the deflection of the spring, as for example axis sections on which the sensor elements are arranged.

The “endoscopic unit” as used herein represents the part of the endodevice which is introduced, fully or partially, into the human body or the like.

The “back end unit” represents the part of the endodevice which serves for steering the endoscopic unit and which is not introduced into the human body or the like.

The term “bendable rod element” refers to a jointed or unjointed respectively elongated deformable structure of the endoscopic unit which comprises at least one degree of freedom. The bendable rod element may be formed out of a continuous material or out of two or more link portions which are connected to one another via one or more joint elements, such as a hinge, a hinge joint, a solid joint, a film hinge and the like.

The material used for the bendable rod element or for two or more the link portions may comprise flexible or resilient characteristics. However, in case of two or more rigid link portions which are connected via joint elements no shape analysis (which would require additional sensing structures) has to performed as compared to a continuous bendable rod element (i.e. due to negligible deformations compared to spring or tendon deformation).

The term “tendon” relates to any sort of cable, string, wire or the like which may be used for bending the bendable rod element and/or tilting link portions about one or more joint elements of the endoscopic unit. The tendons are usually formed of a non-expandable material.

The term “tendon coupler” relates to any sort of coupling means which enable that the tendons of the endoscopic unit may be coupled to and decoupled from the back end unit in an easy and reliable manner. These may be force and/or form fitting connections, as for example click-in or snap-in connections. The tendon usually comprises a corresponding counterpart to the tendon coupler which forms part of the back end unit.

The term “drive member” may be any sort of linear rotational or actuator. Usually, an electric motor is used as drive member.

The term “drive or holding member” relates to structure which may either comprise a linear or rotational actuator such as an electric motor or which may comprise a passive holding respectively receiving means as for example a winch (undriven) or a stationary component of the back end unit to which a (second) spring element is attached with one function end.

The term “sensor element” relates to such devices by means of which the position of an associated axis section may be measured respectively the force/torque which is applied via the tendons to the bendable rod element and/or around a joint element connecting two respective link elements. The sensor elements may be directly part of one of the drive members or motors. The respective sensor signals serve for computing the deflection of the respective spring element.

The term “control unit” refers to a computer device comprising a program respectively an algorithm which is configured to compute on the basis of the sensor signals the deflection of the spring, i.e. which is caused by the movement of the tendons and the respective forces/torques when manipulating the device.

Preferably, the back end unit further comprises a second spring element having a first function end and a second function end, optionally a third sensor element and optionally a fourth sensor element, wherein the second spring element is connectable at its first function end to the second tendon via the second tendon coupler and the second spring element is connected to the drive or holding member at its second function end, wherein between the drive or holding member and the second function end of the second spring element the third sensor element is arranged and between the first function end of the second spring element and the second tendon coupler the fourth sensor element is arranged, wherein each of the third sensor element and the fourth sensor element is configured to generate a sensor signal, and wherein the third sensor element, the fourth sensor element and the drive or holding member are connected to the control unit, wherein the control unit is configured to obtain sensor signals from the third sensor element and the fourth sensor element and to compute a deflection of the second spring element on the basis of the sensor signals obtained from the third sensor element and the fourth sensor element. By providing a second spring element and two additional sensor elements, the endodevice may be operated even more precisely.

Preferably, the first sensor element is associated to a first axis section driven by the drive member and the second sensor element is associated to a second axis section wherein the first spring element connects the first axis section and the second axis section. Thereby, the first sensor element is preferably configured to measure a position of the first axis section and the second sensor element is preferably configured to measure a position of the second axis section. By associating one sensor element to the motor, the motor position may be measured and by associating the second sensor element to the first tendon (respectively a winch on which the tendon is wound up), the load position may be measured. Such placement of the sensor elements enables computing of the deflection of the spring element and therefore the torque applied on the load. It is noted that for measuring the load position, both sensors are needed since the spring element is in between. Only in combination with two sensors, the position of the load is defined, if the tendons do not become slack. Therefore, two tendons with according measurements improve safety.

Preferably, the third sensor element is arranged on a third axis section and the fourth sensor element is arranged on a fourth axis section wherein the second spring element connects the third axis section and the fourth axis section. This arrangement is a precondition for a particularly fine-tuned operation of the endodevice.

Preferably, the third axis section is driven by the drive or holding member. This arrangement enables that each tendon is actively controlled such that precision of the endodevice may still be further enhanced.

Preferably, the third axis section is driven by the drive member via a coupling. Here, one drive member is provided which preferably drives two winches which are mechanically coupled to one another. In this embodiment, the tendons are preferably arranged in opposite movement direction to provide for an efficient handling.

Preferably, the third axis section is supported in a non-driven manner by the drive or holding member. This embodiment provides for one actively controlled tendon and one passive tendon and thus requires less construction effort.

Preferably, the third sensor element is configured to measure a position of the third axis section and the fourth sensor element is configured to measure a position of the fourth axis section. This arrangement again provides for a more precise operation of the endodevice.

Preferably, the drive member comprises an electric motor. By providing an electric motor as drive member, most preferably a DC motor, an efficient control of the first tendon may be provided.

Preferably, the drive or holding member comprises two winches coupled to one another. This embodiment requires only one drive member and two sensor elements for the first spring element (coupled to the active tendon) and thus the least construction effort. The tendons are preferably coupled in the same movement direction. The two winches are preferably coupled mechanically by a common axis section.

Preferably, the drive or holding member comprises an electric motor. By providing an electric motor as drive or holding member, most preferably a DC motor, an efficient control of the first second tendon may be provided.

Preferably, the drive or holding member comprises a stationary supporting structure. The stationary supporting structure may be a component of the back end unit configured to receive an undriven axis section and thus provides for an efficient construction. The embodiment requires one drive member and two sensor elements for the first spring element (coupled to the active tendon) as well as two sensor elements for the second spring element (coupled to the second passive tendon).

Preferably, the drive or holding member comprises a winch which is mechanically coupled to another winch which is driven by the drive member. The embodiment requires one drive member and two sensor elements for the first spring element (coupled to the active tendon) as well as two sensor elements for the second spring element (coupled to the second tendon).

Preferably, the first spring element and/or the second spring element comprise a non-linear spring characteristic curve and in particular a progressive spring characteristic curve. In this manner, the mechanical stiffness of the endoscopic unit joint elements may be varied in an advantageous manner by increasing the tendon pre-tension.

Preferably, the first spring element and/or the second spring element are in the form of a rotational respectively torsional spring. Such spring elements are adoptable to comparatively broad range of surgical applications.

Preferably, the first sensor element, the second sensor element, the third sensor element and/or the fourth sensor element each comprise an encoder. Such senor elements are only little error-prone.

Preferably, the first tendon and the second tendon are configured in the form of a human agonist and antagonist and are preferably attached to a tip portion of the endoscopic unit. Such an arrangement allows for a very precise steering of the endoscopic unit. In case of two joint elements and four tendons, not all of the tendons are attached at the tip portion. Here, the first two tendons are attached after the first joint which they intend to actuate and next two tendons are attached after the second joint which they intend to articulate (i.e. at the tip portion of e.g. a third link element).

Preferably, the first tendon and the second tendon are essentially non-expandable (i.e. the tendons comprise a high structural stiffness). In this manner, more precise position and force measurements for a given set of sensor elements may be achieved.

Preferably, the bendable rod element comprises a first link portion and a second link portion being connected to one another via a first joint element. Such joint elements may comprise hinges or hinge joints, solid joints, film joints and the like. Also, the bendable rod element may comprise a third link portion being connected to the second link portion via a second joint element and which requires additional tendons. The minimum number of tendons depends on the number of degrees of freedom of a joint element. At least two tendons are needed for a first degree of freedom per joint; all other degrees of freedom require at least one additional tendon. Further preferably, the first link portion, the second link portion and/or the third link portion are rigid.

Preferably, an articulated structure is thus provided, i.e. a structure that consists of at least a first and a second rigid link and a dedicated joint element with at least one degree of freedom in between the rigid links. In case of a continuum mechanics structure, the flexibility of the endodevice is based on the compliance of the at least one elongated elastic structure that can deform under external load e.g. from tendon actuation. As a basis for the working principle of the herein endodevice, the shape of the elastic structure of the continuum mechanics structure needs to be measureable with at least the same accuracy as the length change of the actuation mechanism that makes the elongated elastic structure deform.

In case of an articulated robotic endodevice structure, there is no additional shape measurement necessary because the rigid links can be assumed to deform only in a negligible magnitude. A flexible robotic endodevice structure with a mixed design of articulated and continuum mechanics is however possible. For actuation of the at least one degree of freedom per joint or elastic structure, at least a first and a second tendon are required in analogy to human agonist and antagonist. For every additional degree of freedom per joint element (i.e. for joint elements with more than one degree of freedom) at least one further tendon for actuation is required.

Due to a structure of rigid links with dedicated joint elements in or between deformable elastic structures with well-known shape, implementing additional elasticity between the end-effector and the drive allows precise endodevice shape determination, passive endodevice compliance for safety, and joint wrench (i.e. torques and forces) in the degrees of freedom of the joints elements or elastic structure strain and stress measurement.

In this regard, at least two tendons per joint element with one degree of freedom are required, where at least one tendon is actively controlled by a motor while the other at least one tendon is passively actuated by energy storing and releasing elements, e.g. spring elements. For each further actuated/passive degree of freedom per joint element, an actively controlled/passive tendon needs to be added.

Preferably, the tendons that are intended to actuate one joint element are attached to the next link after this joint element, but also more complex actuation strategies where tendons span across several joint elements and jointly actuate those several joint elements can be imagined. At one location along the preferably non-expandable tendon(s), a spring element is introduced that will deform passively under load. The spring element is located outside of the endoscopic unit in the back end unit that can be larger in size since it does not have to be inserted into the patient's body. Before and after each spring element the position of a respective axis section is measured to know the position of the tendon and thus the deflection of the respective spring element.

### BRIEF DESCRIPTION OF THE DRAWINGS

The electro-magnetic induction device according to the invention as well as the inventive method and the inventive system are described in more detail herein below by way of exemplary embodiments and with reference to the attached drawings, in which:

FIG. 1 shows an exploded view of an active subassembly of a backend unit of an inventive endodevice including an electric motor, two encoders and a rotational spring;

FIG. 2 shows a sectional view through an assembled active subassembly of a backend unit of an inventive endodevice according to FIG. 1;

FIG. 3 shows a schematic illustration of an inventive endodevice during use with the endoscopic unit inside a patient's body;

FIG. 4 shows a schematic illustration of an endodevice according to a first exemplary embodiment of the present invention;

FIG. 5 shows a schematic illustration of an endodevice according to a second exemplary embodiment of the present invention;

FIG. 6 shows a schematic illustration of an endodevice according to a third exemplary embodiment of the present invention;

FIG. 7 shows a schematic illustration of an endodevice according to a fourth exemplary embodiment of the present invention;

FIG. 8 shows a schematic illustration of an endodevice according to a fifth exemplary embodiment of the present invention; and

FIG. 9 shows a schematic illustration of an endodevice according to a sixth exemplary embodiment of the present invention.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

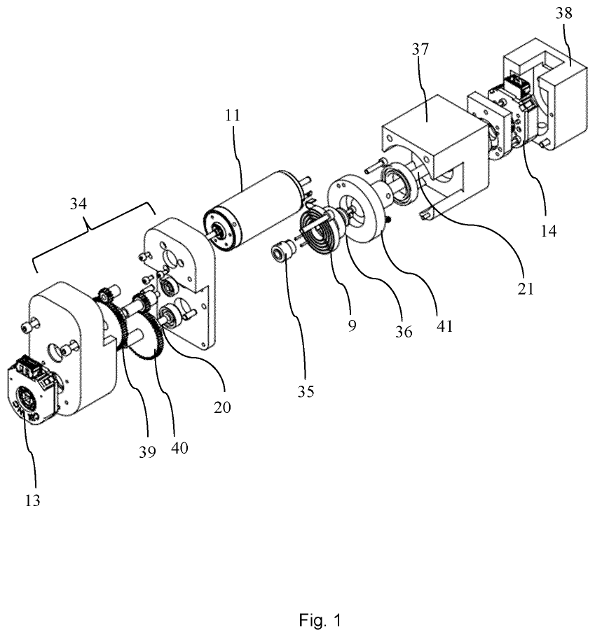

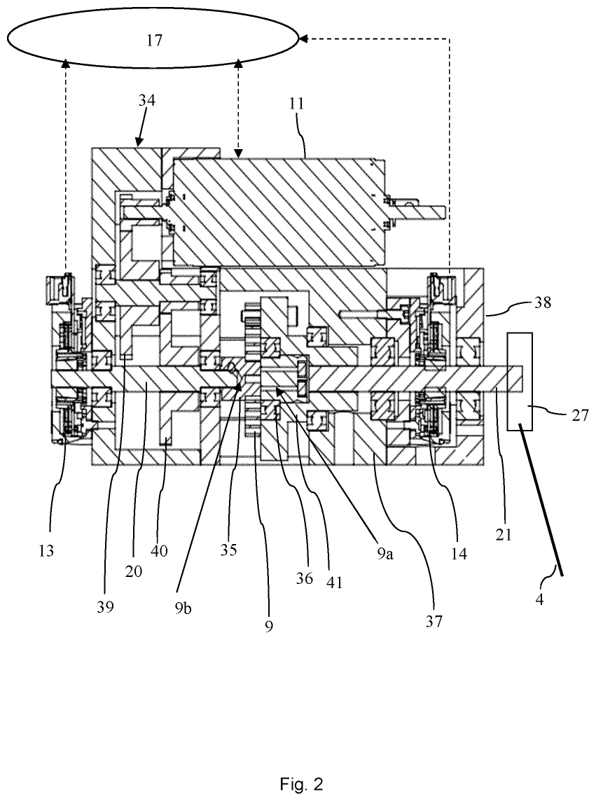

In FIGS. 1 and 2 an “active” subassembly of a backend unit of an inventive endodevice including an electric motor 11, two encoders 13, 14 and a rotational spring element 9. The electric motor 11 drives via gear wheels 39, 40 which are arranged in gear box 34 a first axis section 20. First encoder 13 is arranged on the left end portion of first axis section 20. Below electric motor 11, a first spring element 9 is arranged in a housing part 37. Further, a bearing element 41 of a second axis section 21 is located inside the housing part 37. Second axis section 21 extends through the rear wall of housing part 37 into housing part 38 which houses second encoder 14. Second encoder 14 is arranged on second axis section 21 which further extends through the rear wall of housing part 38. At the right end portion of second axis section 21, a winch 27 is mounted on which a first tendon 4 is wound up (FIG. 2). The first spring element 9 is in the form of a rotational spring which is coupled to the first axis section 20 via first coupling piece 35 and which is coupled to the second axis section 21 via second coupling piece 36. The coupling region of the first spring element 9 with the first axis section 20 (i.e. via the first coupling piece 35) defines the second function end 9b of the first spring element 9. Likewise, the coupling region of the first spring element 9 with the second axis section 21 (i.e. via the first coupling piece 35) defines the first function end 9a of the first spring element 9.

During use of the endodevice, the first encoder 13 measures the position of the first axis section 20 (also referred to as motor position) and the second encoder measures the position of the second axis section 21 (also referred to as load position). This placement of the first and second encoder 13, 14 enables computing of the deflection of the spring element 9 and therefore the torque applied on the load, i.e. on the winch 27 respectively the first tendon 4. The dashed arrows shown in FIG. 2 represent the signals which are provided in this context from the first encoder 13, the second encoder 14 and from the electric motor 11 to the control unit 17 and from the control unit 17 to the electric motor 11.

When computing the desired force for e.g. tendon 4, it is has to be considered that only pulling forces can be exerted on the tendon. To avoid tendon slack (and thus backlash), a minimal pre-tension should be applied to the tendon.

By means of the active subassembly shown in FIGS. 1 and 2, the tendon 4 may be actively controlled via the settings of electric motor 11. In a “passive” subassembly, no electric motor is present but the functional principal of determining the deflection of the respective spring element remains the same, as will be described further below.

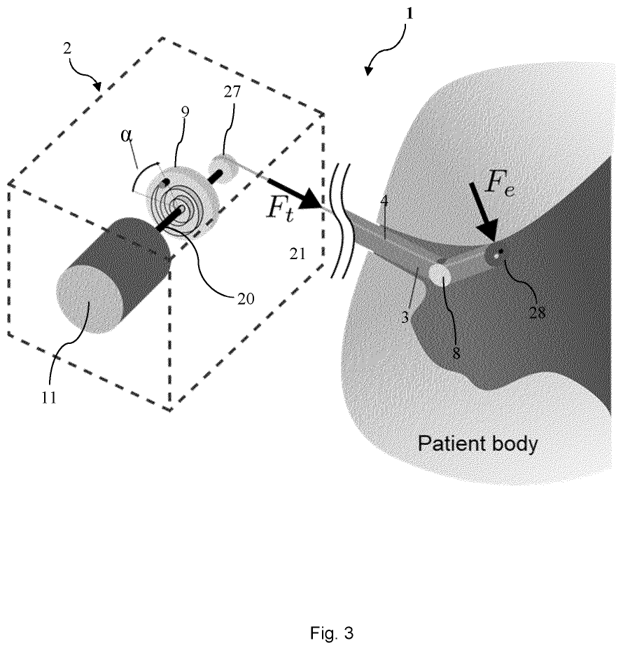

In FIG. 3, an inventive endodevice during use with the endoscopic unit 3 inside a patient's body is schematically illustrated. In the dashed box which shall represent the back end unit 2, again an “active” subassembly is illustrated. Also here, the position of the first axis section 20 (also referred to as motor position) and the position of the second axis section 21 (also referred to as load position) is measured. Therefrom, the deflection a of the spring element 9 and thus the torque/force F t applied via the winch 27 onto the first tendon 4 is computed. Tendon 4 is guided through the endoscopic unit 3 via a first joint element 8 and is finally attached to the tip portion 28 of the endoscopic unit 3. If the tip 28 of the endoscopic unit 3 touches against an inner body respectively tissue portion of the patient, a force F e is exerted on the tip 28 of the endoscopic unit 3 by means of which a haptic feedback may be provided to the user of the endodevice. The haptic feedback provided may be fine-tuned by the varying the stiffness of the spring element respectively by varying the spring characteristics curve.

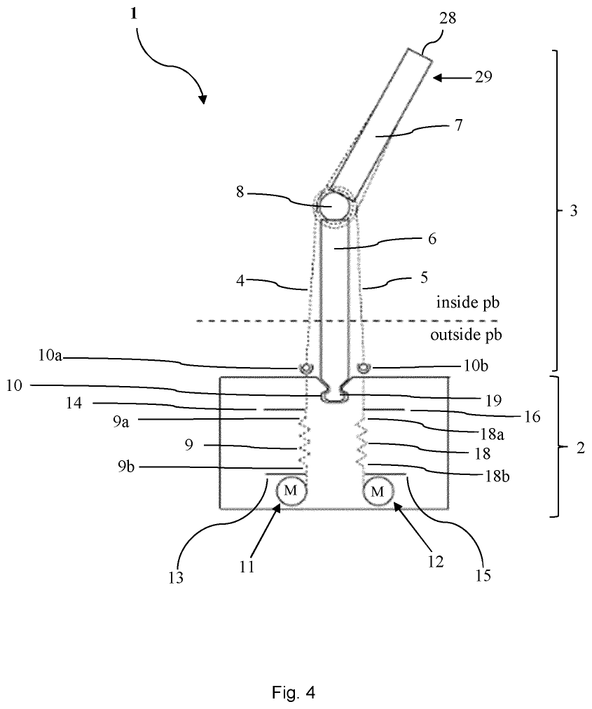

FIG. 4 shows a schematic illustration of an endodevice 1 according to a first exemplary embodiment of the present invention with two actively controlled tendons 4 and 5. The functional principle is the same as described in context with FIGS. 1 to 3. First tendon 4 and second tendon 5 are coupled to the back end unit 2 via first tendon coupler 10a and second tendon couplers 10b. Tendons 4 and 5 are guided through endoscopic unit 3 along bendable rod element 29 which here comprises a first link portion 6 and a second link portion 7 which are connected to one another via a joint element 8. The tendons are guided around joint element 8 and are finally attached to the tip portion 28 of the endoscopic unit 3. The first and second tendon 4, 5 are thereby arranged to act in the form of a human agonist and antagonist. The endoscopic unit 3 is coupled by means of its male coupling structure 19 to the corresponding female coupling structure 10 of the back end unit 2.

The tendon couplers 10a, 10b are each operatively coupled to a respective first function end 9a, 18a of first and second spring elements 9 and 18. Between the tendon couplers 10a, 10b and the first function ends 9a, 18a there are arranged a second and fourth sensor element 14, 16, preferably in the form of an encoder, respectively.

The drive element 11 and the drive and holding element 12 are both in the form of electric motors. Each of the electric motors 11, 12 is operatively coupled to a respective second function end 9b, 18b of first and second spring elements 9 and 18. Between the electric motors 11, 12 and the second function ends 9b, 18b there are arranged a first and third sensor element 13, 15, preferably in the form of an encoder, respectively.

The second function end 9b of the first spring element 9 and the second function end 18b of the second spring element 18 represent the coupling region of the first spring element 9 with the first axis section 20 and, in an analogue manner, a coupling region of the second spring element 18 with a third axis section 22. Likewise, the first function end 9a of the first spring element 9 and the first function end 18a of the second spring element 18 represent the coupling region of the first spring element 9 with the second axis section 21 and, in an analogue manner, a coupling region of the second spring element 18 with a fourth axis section 24.

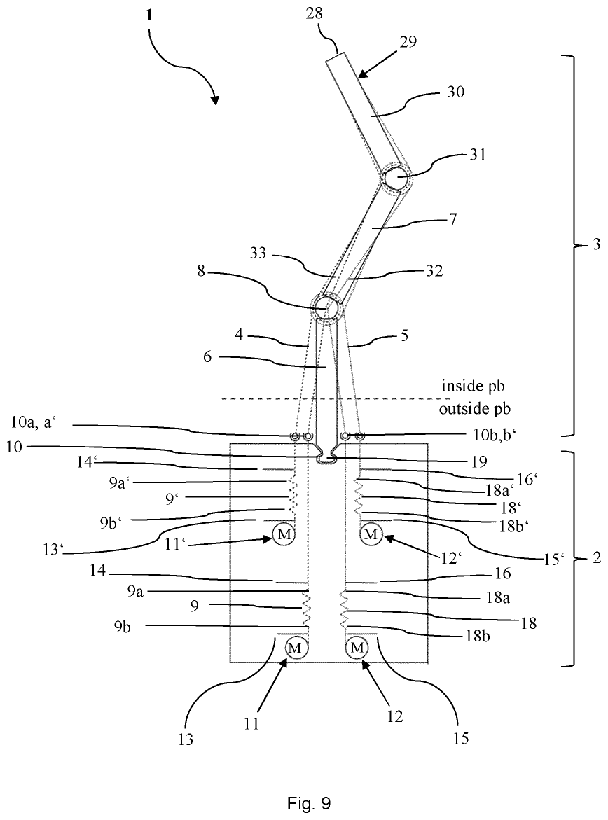

In case of such an embodiment with two independently actuated tendons 4, 5 with two spring elements 9, 18 in series for a one degree of freedom joint element 8a (or bendable rod element 29), choosing a non-linear and preferably progressive spring characteristics curve allows active joint stiffness variation of the actuated degrees of freedom by variation of a pretension of the spring elements 9, 18. For each additional actively controllable degree of freedom in the joint elements 8 (or the bendable rod element 29), an additional independently actuated tendon with a spring element in series is required. Depending on the coupling of the degrees of freedom in one joint element, stiffness selection becomes more or less complex. In case each degree of freedom for one joint element with several degrees of freedom is intended to be controlled independently, always two actively actuated tendons with respective spring elements should be foreseen, as described further below in connection with FIG. 9.

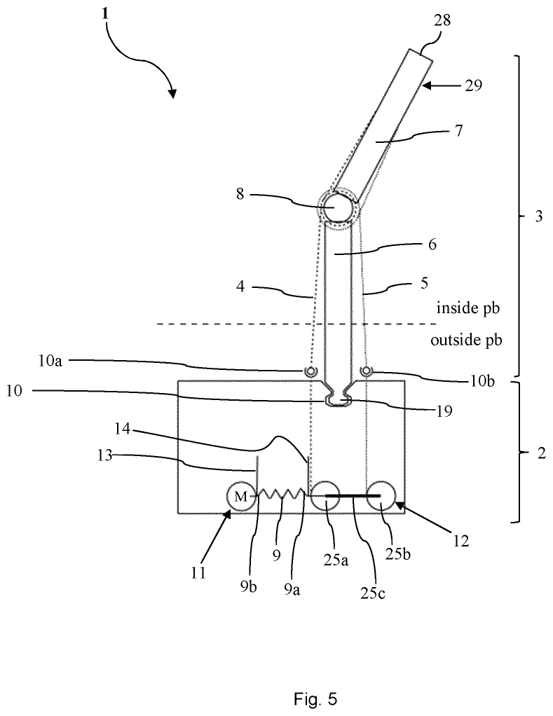

FIG. 5 shows a schematic illustration of an endodevice 1 according to a second exemplary embodiment of the present invention with one actively controlled tendon 4 and one passive tendon 5. Except from the configuration of the drive or holding member 12, the endodevice 1 essentially corresponds to the principles described in the previous figures.

Here, the drive or holding member 12 comprises a first winch 25a and a second winch 25b with a common axis 25c which connects winches 25a, 25b (also a “double winch” may be provided for the two tendons on axis 25c) and there is coupling to a second spring element. The first tendon 4 is wound up on first winch 25a and the second tendon 5 is wound up on second winch 25b wherein the tendons 4, 5 comprise the same movement direction. The common axis 25c comprises the function of the second axis section 21 as shown and described in the previous figures. In other words, the first function end 9a of the first spring element 9 represents the coupling region of the first spring element 9 with the common axis 25c. This results in a simple construction with passive control of the second tendon 5 with only two sensor elements 13, 14 for position measuring.

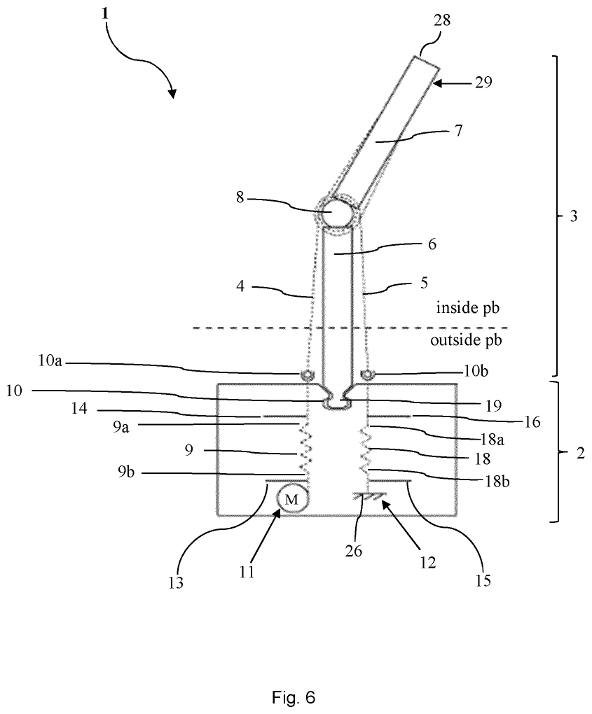

FIG. 6 shows a schematic illustration of an endodevice 1 according to a third exemplary embodiment of the present invention with one actively controlled tendon 4 and one passive tendon 5. Except from the configuration of the drive or holding member 12, the endodevice 1 essentially corresponds to the principles described in the previous figures.

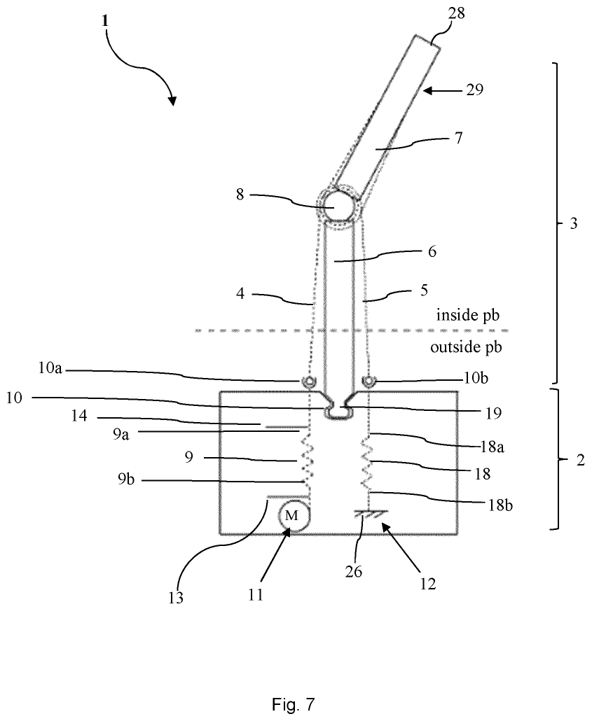

Here, the drive or holding member 12 comprises a passive stationary supporting structure 26, i.e. instead of a second electric motor as in case of the endodevice shown in FIG. 4. The second function end 18b of the second spring element 18 represent the coupling region of the second spring element 18 with a third axis section 22 wherein the third axis section 22 is supported, in an undriven manner, in the stationary supporting structure 26. The stationary supporting structure can for example be in the form of a housing part having an axis bearing. This also provides for a comparatively simple construction with passive control of the second tendon 5, however with two spring elements 9, 18 and four sensor elements 13, 14, 15, 16 for position measuring. It is also possible to omit sensor elements 15, 16 before and after the second spring element 18, as shown in the fourth embodiment according to FIG. 7. With such construction a slightly better control of the passive second tendon may be achieved, respectively.

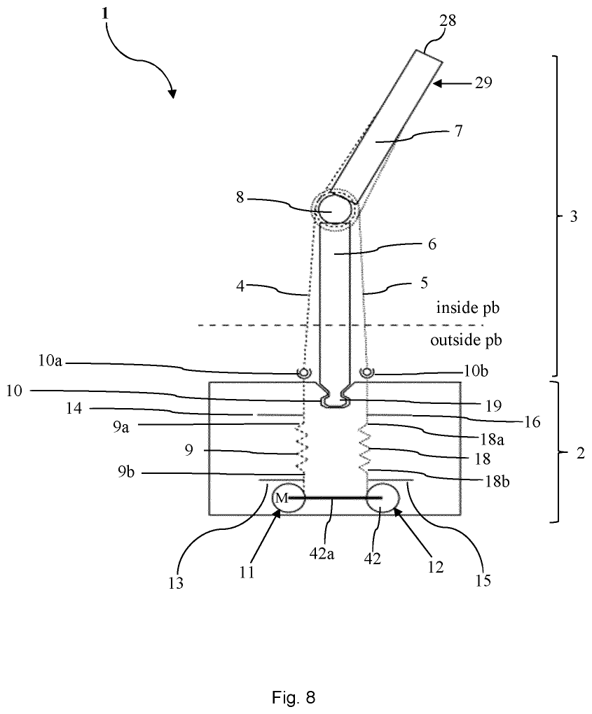

FIG. 8 shows a schematic illustration of an endodevice 1 according to a fifth exemplary embodiment of the present invention where the first tendon 4 and the second tendon 5 are arranged in opposite movement direction through mechanically coupled winches. Except from the configuration of the drive or holding member 12, the endodevice 1 essentially corresponds to the principles described in the previous figures.

Here, the drive or holding member 12 comprises a winch 42 which is coupled via axis 42a with a winch relating to electric motor 11. The tendons 4, 5 are thus arranged in opposite movement directions. The first tendon 4 and the second tendon 5 are thus actuated by the same electric motor 11.

The second function end 9b of the first spring element 9 and the second function end 18b of the second spring element 18 represent the coupling region of the first spring element 9 with the first axis section 20 and, in an analogue manner, a coupling region of the second spring element 18 with a third axis section 22 which is driven by electric motor 11 via axis 42a.

FIG. 9 shows a schematic illustration of an endodevice 1 according to a sixth exemplary embodiment of the present invention with four actively controlled tendons 4, 32 and 33. The functional principle is the same as described in context with FIGS. 1 to 4.

First tendon 4, second tendon 5, third tendon 32 and fourth tendon 33 are coupled to the back end unit 2 via first tendon coupler 10a, second tendon couplers 10b, third tendon coupler 10a′ and fourth tendon coupler 10b′. The bendable rod element 29 here comprises a first link portion 6, a second link portion 7 and a third link portion 30 which are connected to one another via a first joint element 8 and a third joint element 31. The tendons 4, 5 are guided around first joint element 8 and are attached at a top portion of second link element 7. Tendons 32 and 33 are guided around first joint element 8 and are attached at the tip portion 28 of the endoscopic unit 3.

The tendons 4, 5 and 32, 33 are thereby arranged to act in the form of a human agonist and antagonist, respectively. The endoscopic unit 3 is coupled by means of its male coupling structure 19 to the corresponding female coupling structure 10 of the back end unit 2.

The tendon couplers 10a, 10b, 10a′ and 10b′ are each operatively coupled to a respective first function end 9a, 9a′ 18a and 18a′ of first, second, third and fourth spring elements 9, 9, 18 and 18′. Between the tendon couplers 10a, 10a′, 10b and 10b′ and the first function ends 9a, 9a′, 18a and 18a′ there are arranged a second and fourth sensor elements 14, 14′, 16 and 16′, preferably in the form of an encoders, respectively.

The drive elements 11, 11′ and the drive and holding element 12, 12′ are each in the form of electric motors. Each of the electric motors 11, 11′ 12, 12′ is operatively coupled to a respective second function end 9b, 9b′ 18b, 18b′ of first and second spring elements 9, 9′, 18 and 18′. Between the electric motors 11, 11′, 12 and 12′ and the second function ends 9b, 9b′ 18b and 18b′ there are arranged first and third sensor elements 13, 13′, 15 and 15′, preferably in the form of an encoder, respectively.

The second function ends 9b, 9b′ of the first and third spring elements 9, 9′ and the second function ends 18b, 18b′ of the second and fourth spring element 18, 18′ represent the coupling regions of the first and third spring elements 9, 9′ with first axis sections 20, 20′ and, in an analogue manner, a coupling region of the second and fourth spring elements 18, 18′ with a third axis sections 22, 22′. Likewise, the first function ends 9a, 9a′ of the first and third spring elements 9, 9′ and the first function end 18a, 18a′ of the second and fourth spring elements 18 represent the coupling region of the first and third spring elements 9, 9′ with the second axis section 21, 21′ and, in an analogue manner, a coupling region of the second and fourth spring element 18, 18′ with a fourth axis section 24, 24′.

This description and the accompanying drawings that illustrate aspects and embodiments of the present invention should not be taken as limiting—the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the figures individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

### LIST OF REFERENCE NUMBERS

1 endodevice
2 back end unit
3 endoscopic unit
4 first tendon
5 second tendon
6 first (link) portion
7 second (link) portion
8 joint element
9 first spring element
9′ third spring element
9a first function end
9a′ first function end
9b second function end
9b′ second function end
10 back end coupling structure
10a first tendon coupler
10a′ third tendon coupler
10b second tendon coupler
10b′ fourth tendon coupler
11 drive member (electric motor)
11′ second drive member (electric motor)
12 drive or holding member (electric motor or passive structure)
12′ second drive or holding member (electric motor or passive structure)
13 first sensor element
13′ fifth sensor element
14 second sensor element
14′ sixth sensor element
15 third sensor element
15′ sept sensor element
16 fourth sensor element
16′ eighth sensor element
17 control unit
18 second spring element
18′ fourth spring element
18a first function end
18a′ first function end
18b second function end
18b′ second function end
19 endoscopic coupling structure
20 first axis section
20′ first axis section
21 second axis section
21′ second axis section
22 third axis section
22′ second axis section
23 fourth axis section
23′ fourth axis section
24 coupling
25a winch
25b winch
25c axis
26 stationary supporting structure
27 winch
28 tip portion endoscopic unit
29 bendable rod element
30 third link portion
31 second joint element
32 third tendon
33 fourth tendon
34 gear box
35 first coupling piece
36 second coupling piece
37 housing part (spring element)
38 housing part (encoder)
39 gear wheel
40 gear wheel
41 connection element
42 winch
42a axis
α spring deflection

1. An endodevice with tension control, the endodevice comprising:
a back end unit; and
an endoscopic unit configured to be coupled to the back end unit,
wherein the endoscopic unit includes
an endoscopic coupling structure,
a bendable rod element, and
a first tendon and a second tendon arranged to bend a first portion of the bendable rod element and a second portion of the bendable rod element relative to one another,
wherein the back end unit includes
a back end coupling structure corresponding to the endoscopic coupling structure and including a first tendon coupler and a second tendon coupler,
a first spring element having a first function end and a second function end, and
a drive member and a drive or holding member,
wherein the first spring element is connectable at its first function end to the first tendon via the first tendon coupler and the first spring element is connected to the drive member at its second function end,
wherein the drive or holding member is connectable to the second tendon via the second tendon coupler,
wherein the back end unit further comprises a first sensor element and a second sensor element,
wherein between the drive member and the second function end of the first spring element the first sensor element is arranged and between the first function end of the first spring element and the first tendon coupler the second sensor element is arranged,
wherein each of the first sensor element and the second sensor element is configured to generate a sensor signal,
wherein the first sensor element, the second sensor element and the drive member are connected to a control unit, and
wherein the control unit is configured to obtain sensor signals from the first sensor element and the second sensor element and to compute a deflection of the first spring element on the basis of the sensor signals obtained from the first sensor element and the second sensor element.

2. The endodevice according to claim 1, wherein the back end unit comprises:
a second spring element having a first function end and a second function end,
a third sensor element and
a fourth sensor element,
wherein the second spring element is connectable at its first function end to the second tendon via the second tendon coupler and the second spring element is connected to the drive or holding member at its second function end wherein between the drive or holding member and the second function end of the second spring element the third sensor element is arranged and between the first function end of the second spring element and the second tendon coupler the fourth sensor element is arranged,
wherein each of the third sensor element and the fourth sensor element is configured to generate a sensor signal,
wherein the third sensor element, the fourth sensor element and the drive or holding member are connected to the control unit, and
wherein the control unit is configured to obtain sensor signals from the third sensor element and the fourth sensor element and to compute a deflection of the second spring element on the basis of the sensor signals obtained from the third sensor element and the fourth sensor element.

3. The endodevice according to claim 1, wherein the first sensor element is associated to a first axis section driven by the drive member and the second sensor element is associated to a second axis section wherein the first spring element connects the first axis section and the second axis section.

4. The endodevice according to claim 3, wherein the first sensor element is configured to measure a position of the first axis section and the second sensor element is configured to measure a position of the second axis section.

5. The endodevice according to claim 2, wherein the third sensor element is arranged on a third axis section and the fourth sensor element is arranged on a fourth axis section wherein the second spring element connects the third axis section and the fourth axis section.

6. The endodevice according to claim 5, wherein the third axis section is driven by the drive or holding member, wherein the third axis section is driven by the drive member via a coupling, or wherein the third axis section is supported in a non-driven manner by the drive or holding member.

7. (canceled)

8. (canceled)

9. The endodevice according to claim 5, wherein the third sensor element is configured to measure a position of the third axis section and the fourth sensor element is configured to measure a position of the fourth axis section.

10. The endodevice according to claim 1, wherein the drive member comprises an electric motor.

11. The endodevice according to claim 1, wherein the drive or holding member comprises two winches coupled to one another.

12. The endodevice according to claim 2, wherein the drive or holding member comprises an electric motor.

13. The endodevice according to claim 2, wherein the drive or holding member comprises a stationary supporting structure.

14. The endodevice according to claim 2, wherein the drive or holding member comprises a winch which is coupled to the drive member.

15. The endodevice according to claim 1, wherein the first spring element and/or the second spring element comprise a non-linear spring characteristic curve.

16. The endodevice according to claim 1, wherein the first spring element and/or the second spring element are in the form of a rotational spring.

17. The endodevice according to claim 2, wherein the first sensor element, the second sensor element, the third sensor element and/or the fourth sensor element each comprise an encoder.

18. The endodevice according to claim 1, wherein the first tendon and the second tendon are configured in the form of a human agonist and antagonist and are preferably attached to a tip portion of the endoscopic unit.

19. The endodevice according to claim 1, wherein the first tendon and the second tendon are essentially non-expandable.

20. The endodevice according to claim 1, wherein the bendable rod element comprises a first link portion and a second link portion being connected to one another via a first joint element.

21. The endodevice according to claim 20, wherein the bendable rod element comprises a third link portion being connected to the second link portion via a second joint element.

22. The endodevice according to claim 21, wherein the first link portion, the second link portion and/or the third link portion are rigid.
